(19) United States
(12) Patent Application Publication
(10) Pub. No.: US 2026/0250726 A1
(43) Pub. Date: Aug. 27, 2026

(54) BIOFUEL PRODUCTION

(71) Applicant: Horizon Bioinnovation AB, Lund (SE)

(72) Inventor: Gashaw MAMO, Lund (SE)

(21) Appl. No.: 19/165,645

(22) PCT Filed: Mar. 15, 2024
(86) PCT No.: PCT/EP2024/057088

§ 371 Date: Sep. 16, 2025

(30) Foreign Application Priority Data
Mar. 17, 2024 (SE) 2350300-6

(51) Int. Cl. C12P 7/649; C10L 1/02; C11B 1/02; C11B 1/10

(52) U.S. Cl. CPC C12P 7/649; C10L 1/02; C11B 1/025; C11B 1/10; C10L 2200/0469

(57) ABSTRACT

Disclosed is a method of lipid extraction for biofuel production comprising treating an ethanol-biomass mixture with ozone and/or at least one protease, followed by extraction of the ethanol fraction with an immiscible organic solvent. Also disclosed is a process for biofuel production comprising culturing psychrophilic and alkaliphilic microalgae, harvesting the microalgae and extracting lipids from the harvested cells. Also disclosed is lipid biofuel from microalgae, characterized by reduced chlorophyll content.

### FIELD

The disclosure relates to biofuel production from biomass, in particular novel methods of extracting lipids from autotrophic microbes.

### INTRODUCTION

The need of energy is increasing continuously worldwide due to a constant increase in industrialization and population. The basic sources of the energy are petroleum, natural gas, coal, hydro and nuclear. A major disadvantage of using petroleum based fuels is atmospheric pollution created by their combustion that results in a major source of greenhouse gas, in particular CO2.

Large-scale introduction of biomass energy could contribute to sustainable development on several fronts, environmentally, socially and economically. Certain microbes such as microalgae capture carbon dioxide from the atmosphere through photosynthesis. These microbes can be obtained easily from a variety of marine and terrestrial environments in large quantities, thereby providing important life resource in renewable energy technology, especially biodiesel production.

Although any microbial biomass that has the ability to accumulate lipids (so-called oleogenic microbes) can be useful for biofuel production, microalgae have been found to be particularly useful for this purpose. Microalgae comprise a vast group of unicellular photosynthetic organisms that have an extraordinary potential for cultivation as energy crops. Microalgae are furthermore a great source of many highly valuable products such as polyunsaturated fatty acids, astaxanthin and bioactive compounds. Production of oil from microalgae has been described in US Patent Application No. 20080160593.

The process of biofuel production requires the separation of the biomass lipids from the microbes themselves. This has proven to represent a formidable challenge, involving typically the rupturing of the microbial cells, followed by isolation of the lipids from the ruptured cells. Moreover, the process typically involves the drying of microbial mass prior to lipid extraction, requiring an excessive amount of energy.

### SUMMARY

The present invention provides methods to overcome, eliminate or mitigate deficiencies of the prior art, for example the deficiencies described in the above.

In an aspect, the invention relates to a method of lipid extraction for biofuel production. The method comprises steps of (a) providing a wet oleogenic microbial mass; (b) adding ethanol to the wet oleogenic microbial biomass to obtain an ethanol-biomass mixture; (c) treating the ethanol-biomass mixture from step (b) with ozone and/or one or more protease; (d) separating insoluble material present in the ethanol-biomass mixture to obtain an ethanol-containing soluble fraction; (e) extracting the ethanol-containing fraction with an organic solvent; and (f) removing the organic solvent to obtain extracted microalgal lipid.

The addition of ethanol can be preceded by the addition of an alkaline solution containing a suitable base such as NaOH or KOH. It can be preferable to treat the ethanol-biomass mixture in step (b) with both ozone and one or more protease.

The wet biomass contains a certain amount of water, i.e. the biomass is not a dry or dehydrated form. The wet biomass can comprise whole cells or the biomass can comprise homogenized or ruptured cells, or cellular fragments obtained from such rupturing or homogenization.

Another aspect relates to a process for biofuel production that comprises steps of (i) selecting oleogenic microalgae that have an optimal growth at a temperature of about 20° C. or lower and a pH of 8 or higher; (ii) culturing the oleogenic microalgae in an aqueous culture medium in the presence of sunlight, nutrients and CO₂; (iii) harvesting oleogenic microalgal cells obtained from the culturing in step (ii); and (iv) extracting lipids from the harvested oleogenic microalgal cells obtained in step (iii) to obtain lipid biofuel.

The extracting in step (iv) can preferably be performed by a lipid extraction method as described in the above, i.e. a method comprising steps of (a) providing a wet oleogenic microbial mass; (b) adding ethanol to the wet oleogenic microbial biomass to obtain an ethanol-biomass mixture; (c) treating the ethanol-biomass mixture from step (b) with ozone and optionally one or more protease; (d) separating insoluble material present in the ethanol-biomass mixture to obtain an ethanol-containing soluble fraction; (e) extracting the ethanol-containing fraction with an organic solvent; and (f) removing the organic solvent to obtain extracted microalgal lipid.

Another aspect relates to lipid biofuel from oleogenic microalgae, the lipid biofuel characterized by a chlorophyll content that is less than 500 ppm. The oleogenic microalgae can be psychrophilic and/or alkaliphilic. For example, the microalgae can have an optimal growth at a temperature of about 20° C. or lower and a pH of 8 or higher.

Yet another aspect relates to oleogenic microalgae that have an optimum growth at a temperature of about 20° C. or lower and pH of 8 or higher.

### BRIEF DESCRIPTION OF THE DRAWINGS

FIG. 1 shows in (A) a schematic diagram of a lipid extraction process; in (B) the process includes a pretreatment step with NaOH.

FIG. 2 shows the effects of protease treatment.

FIG. 3. Shows the difference in the pigmentation of the extracted lipid when biomass is treated with NaOH. The lipid extract on left (A) is obtained using FIG. 1A process and the one on the right (B) is from the FIG. 1B process.

FIG. 4 shows a comparison of the yield of lipid extraction for the different processes in FIG. 1 as well as comparison to yield from dried biomass.

### DESCRIPTION

The present invention relates to biofuel production from oleogenic microbes, and methods for extracting lipids therefrom. In particular applications, the invention relates to use of cold-adapted (psychrophilic) microbial strains that can grow at low temperatures, e.g. at 20° C. or lower. Preferably, the strains are also alkaliphilic, providing for opportunities to culture the strains using natural CO₂ in ambient air only, due to the increased solubility of CO₂ at elevated pH. In other words, there is no need to supplement the naturally dissolved CO₂ by additional sources of CO₂.

The microbes can be any oleogenic microbes, i.e. microbes that can accumulate increased amounts of lipids. Such microbes can be used in biotechnology to obtain lipids by extraction processes. Oleogenic microbes can accumulate lipids to represent a significant portion (up to 20-70%) of their total cell mass depending on their growth conditions. Thus, both nutrition and temperature can significantly influence the degree of lipid accumulation. For example, it has been found that lipid accumulation increased significantly in yeast at decreased temperatures.

The oleogenic microbes can in general be from one or more yeast, bacteria or microalgae species. In certain applications, the microbes are microalgae. The microalgae can be cold-adapted (psychrophilic) microalgae and/or microalgae that are capable of growing at elevated pH (e.g., at a pH of 8 or greater).

The term “psychrophilic” as disclosed herein refers to microbes that have an optimal temperature for growth of 20° C. or lower.

Psychrophilic microbes can withstand temperatures as low as 0° C., meaning that the microbes are capable of growth at a temperature of 0° C. The microbes can generally withstand a temperature as high as 25° C.

Psychrophilic microbes as disclosed herein can accordingly grow at a temperature in the range of 0° C. to 25° C., including 0° C. to 20° C., 5° C. to 20° C. and 10° C. to 20° C. The optimum growth temperature of the microbes can be in the range of 10° C. to 25° C., such as in the range of 10° C. to 20° C. or 15° C. to 20° C.

The psychrophilic microbes are preferably also alkaliphilic, i.e. the microbes are capable of growth under alkaline conditions. Preferably, the microbes can grow at a pH in the range from about 8 to 13, such as about 8.5 to about 13, about 8.5 to about 12 or about 8.5 to about 11.

Open pond cultivation is a particulary promising field of growing microbes for biofuel production, since such cultivation is relatively inexpensive and is easily scalable.

Moreover, open pond cultivation can be performed independent of geographical location, not requiring use of valuable farmland.

Cultivation of microalgae in open ponds is normally performed under autotrophic conditions, i.e. utilizing photosynthetic carbon fixation and using mesophilic microbes that can grow under normal conditions of temperature and pH. Since dissolved CO₂ and HCO₃⁻ is in low concentration in water at neutral pH, there needs to be supplemental CO₂ provided for growth, which is normally done through by delivering additional/supplementary CO₂ into the pond or through use of flue gas. This however results in significant increase in cost of open pond culturing.

The solubility of CO₂ in water increases exponentially with pH above neutral pH. As a consequence, dissolved CO₂ in water is almost 4 orders of magnitude greater at pH 10 compared with pH 6. This in turn means that microbes that can grow at elevated pH can use dissolved CO₂ as a carbon source without need of supplying addition CO₂. This also means that microbial growth utilizing atmospheric CO₂ that naturally dissolves in alkaline environment is a great way to harvest CO₂, thereby representing one tool to combat increased CO₂ emissions and resulting global warming.

The open pond culturing, performed at elevated pH, can thus generally be performed without supplementing the naturally occurring dissolved CO₂ in the pond environment. The open pond can be a natural or artificial pond or a raceway pond.

The culturing can involve addition or use of an inorganic buffer to maintain the pH in the open pond at a predetermined level. The inorganic buffer can be any suitable buffer, including for example carbonate buffers (e.g., NaHCO₃/Na₂CO₃ and/or KHCO₃/K₂CO₃). The concentration of such inorganic buffer can generally range from about 5 mM to about 1M, such as from about 10 mM to about 500 mM, from about 10 mM to about 250 mM or from about 10 mM to about 100 mM.

It may be useful to provide circulation and/or mixing within the open pond. Thus, the cultivation method can involve circulating the microalgae within the open pond. Such circulation can be performed using methods known in the art, such as by forced circulation using on more pumping means, for example vertical (fountain) pumping, submerged pumping, paddlewheel pumping. The circulation can also be affected by propeller pumping or through air lifting, using air blowing. Any combination of such circulation means can also be used.

Lipid extraction from oleogenic microbes traditionally involves a drying step of the wet biomass, since lipid extraction is generally found to be more efficient from dry biomass. Traditional lipid extraction process therefore involve cultivation, dewatering followed by drying in hot oven, lipid extraction, removal of cell debris and removal of solvent by distillation.

However, although the lipid extraction is more favorable, the process requires enormous use of energy to dry the biomass. This has rendered lipid extraction from biomass economically prohibitive for efficient and competitive biofuel production.

The present invention provides a process that allowes efficient extraction of lipids from wet biomass, thereby providing enormous advantages over current extraction methods, by eliminating the need for drying the biomass prior to treatment to obtain biofuel. The process is robust and easily scalable for industrial biofuel production.

Overall, the lipid extraction is based on the chemical concept that “like dissolves like”. The lipid extraction starts from a wet biomass that can include whole cells, cellular fragments (ruptured/homogenized cells), or a composition of partially ruptured and/or homogenized cells. The biomass can be from any oleogenic microbe or mixture of two or more microbes. The microbes can be microalgae, such as psychrophilic and/or alkaliphilic microalgae.

Microalgal cultivation mainly follows autotrophic growth. Almost all microalgae are photosynthetic in nature having chlorophyll a, chlorophyll b and bacterio-chlorophyll in some blue-green algae (cyanobacteria), and thus are significant solar energy convertors. As a consequence, the microalgae are cultivated in illuminated environments either in open or closed cultivation systems. Cultivation of microalgae for lipid biofuel production aims at maximizing the lipid productivity along with the growth rate of the microalgae.

Accumulation of lipids in microalgae can be attributed to the consumption of sugars at a rate higher than that of the rate of cell doubling, which promotes conversion of excess sugar into lipids which favor the algae in their stationary phase of growth to fight the nutrient depletion.

Oleogenic microalgae useful in the methods described herein can be from one or more genus selected from Chlorella, Nannochloropsis, Chlamydomonas, Scenedesmus, Synechocystis, Tetraselmis, Monoraphidium, Ostreococcus, Koliella, Pseudopleurochloris sp Tisochrysis and Phaeodactylum. Exemplary useful species are Chlorella vulgaris, Chimydomonas reinhardtii, Tetraselmis chuii, Tetraselmis suecica, Nannochloropsis gaditana. For example, the microalgae can be from one or more species selected from Chlamydomonoas sp, Tetraselmis sp or Chlorella sp. In certain embodiments, the species is Chlorella vulgaris.

The first extraction step involves use of a solvent that can solubilize the lipids to allow separation of lipids from cellular fragments and precipitated protein. The solvent can be a polar solvent, such as an alcohol. Preferably, the solvent is ethanol. The solvent can alternatively comprise one or more hydrocarbon solvent.

The biomass is a wet biomass with liquid content that can be in the range of 1-20 mL/g, such as 1-10 mL/g or 2-10 mL/g of dry biomass.

Ozone is a known disinfectant and used for example to regulate smell/taste and biological growth. The present inventors have discovered that the treatment of liquid biomass mixture with ozone has been found to result in improved extraction efficiency.

Treatment with ozone generally involves bubbling ozone gas through the solution. The ozone treatment can generally be done at a temperature in the range of about 0° C. to about 40° C., such as about 5° C. to about 30° C., about 10° C. to about 25° C., or about 15° C. to about 25° C. The treatment can also be done at room temperature.

The treatment may be performed for a period of time in the range of about 1 to 60 minutes, such as about 2 to 60 minutes, about 5 to about 60 minutes, about 10 to about 60 minutes, about 20 to about 60 minutes about 20 to about 40 minutes or about 20 to about 40 minutes.

The flow rate of ozone gas can be in the range of 0.2 to 2g ozone for each g of biomass per hour, such as in the range of 0.5 to 1.5g or 0.8 to 1.2g.

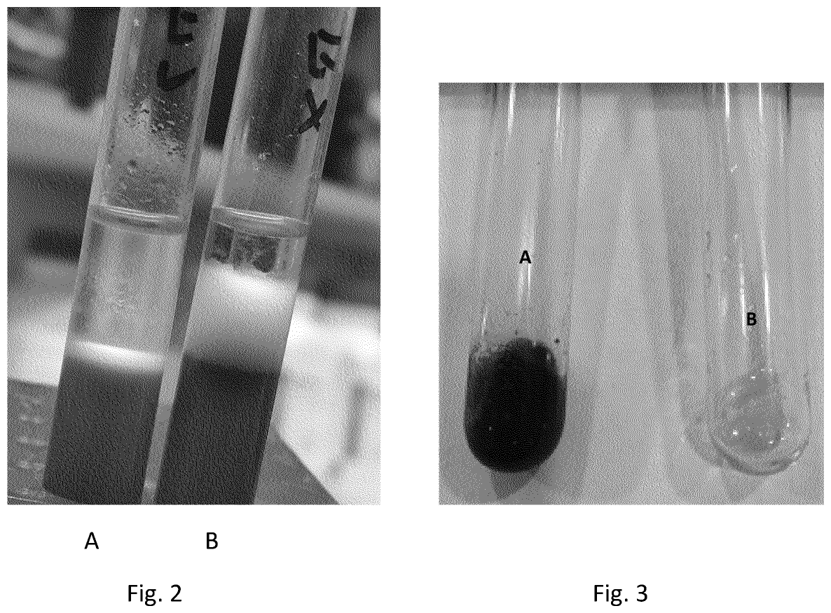

Protease treatment has been found to result in diminished protein content and emulsion formation which results in improved lipid quality and increased yield of lipid. The effects of protease treatment of the formation of emulsion are illustrated in FIG. 2, showing that by including protease, the cellular mass and precipitated protein/carbohydrate are clearly separated from the lipid-containing liquid fraction, thereby providing for easy separation of the material.

The protease can be any suitable industrial protease known in the art, such as a microbial protease. For example, the protease can be an alkaline protease from a suitable species such as Bacillus sp. Alternatively, the protease can be a an acidic or neutral protease. The protease can also be of animal origin, such as trypsin, pepsin or chymotrypsin. A combination of two or more proteases of microbial and/or animal origin can also be used.

The process can generally include treatment with one or more protease, treatment with ozone, or treatment both with ozone and one or more protease. A combination of treatment with ozone and protease has therefore been found to particularly advantageous, resulting in improved extraction efficiency, thereby resulting in an increased quality and yield of the final extracted lipid material.

Ozone and protease treatment can be done concomitantly, i.e. the protease can be added to the solution and the resulting solution treated with ozone. The protease can also be added to a solution through which ozone is being bubbled, and the protease treatment allowed to continue while ozone treatment is ongoing.

Alternatively, the ozone treatment can precede protease treatment or vice versa. Thus, the solution can first be treated with ozone, and subsequently with protease. Alternatively, the solution can be treated first with protease, followed by treatment with ozone.

The biomass is separated from the soluble fraction by any common means, such as filtration, centrifugation or sedimentation, or any combination of such techniques. The biomass thus obtained can be used for bioethanol production, utilizing the high carbohydrate content of the biomass. Alternatively, the biomass can be used for other purposes, such as in the production of feed or food, in particular when the biomass is from a nutritious organism such as microalgae.

The liquid fraction is subsequently reduced in volume, e.g. by distillation. When ethanol is used as solvent, the thus obtained ethanol can be recovered and used for other purposes or reused in in the process.

The microbial lipids can be present in micellar form or in solution in the remaining concentrated organic solvent (e.g. ethanol) fraction. To collect the lipids, a second extraction step is performed. The solvent used for the extraction should be a suitable non-polar or polar solvent that is immiscible with the first solvent. Exemplary solvent include solvents such as hexane and ethyl acetate. The skilled person will appreciate that alternative suitable solvents may be used, as long as the solvent can serve the purpose of (i) being able to extract the lipids and (ii) be easily removable.

The second extraction step is followed by removal of the extraction solvent (e.g., ethyl acetate or hexane), e.g. by distillation. The solvent thus obtained can be collected and reused or recycled for other purpose.

The solvent removal results in a lipid fraction that is pure and substantially free from protein and carbohydrates.

The invention further relates to microalgal biofuel, i.e. biofuel containing lipids from oleogenic microalgae.

The presence of chlorophyll is known to decrease the transesterification and combustion efficiency of biofuel. Moreover, chlorophyll leads to oxidative decay of the biofuel. In short, the presence of chlorophyll decreases the quality of biofuel and hence it is important to remove the chloropyll at least partially.

Different techniques such column chromatography, bleaching treatment with activated diatomite (e.g. at 80° C. for 2 h), treatment with hydrogen peroxide (e.g. for about 1 h), and treatment with addition of H₃PO₄ (e.g. about 1%) and heating (e.g. for 80° C. for about 30 min) can be used to remove chlorophyll, alone or in combination. However, such treatment is an extra step and incurs some cost and time.

As shown in FIG. 3, treatment of wet biomass with alkali (0.1 to 1M NaOH) before the addition of ethanol, followed by extraction of the lipid results in a low amount of chlorophyll in the final lipid material (less than 10% of the original chlorophyll).

Accordingly, the extraction process can include one or more pretreatment steps to remove or reduce chlorophyll in the biomass. The pretreatment can be done by treatment of wet biomass with base such as NaOH or KOH, for example an aqueous solution with base concentration in the range of about 0.1M to about 2M, such as about 0.1M to about 1M. The pretreatment can be effected by the addition of an aqueous solution containing the base in this concentration to the wet biomass.

The present disclosure also provides lipid biofuel from microalgae that has a reduced chlorophyll content. The lipid biofuel can have a chlorophyll content that is less than about 500 ppm, such as less than about 400 ppm, less than about 300 ppm, less than about 200 ppm or less than about 100 ppm. The lipid biofuel can be obtained by a process as disclosed herein.

The chlorophyll content can be less than about 10% of the original chlorophyll content in the biomass, i.e. less than 10% of the chlorophyll content in untreated biomass, such as less than about 8%, less than about 6% or less than about 5%.

Accordingly, the lipid biofuel has improved combustion efficiency compared with conventional lipid biofuel obtained from microalgae and better storage stability.

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to” and are not intended to exclude other components.

Those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure are used. The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

The term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

Features disclosed in the specification, unless stated otherwise, can be replaced by alternative features serving the same, equivalent or similar purpose. Thus, unless stated otherwise, each feature disclosed represents one example of a generic series of equivalent or similar features.

Use of exemplary language, such as “for instance”, “such as”, “for example” and the like, is merely intended to better illustrate the invention and does not indicate a limitation on the scope of the invention unless so claimed. Any steps described in the specification may be performed in any order or simultaneously, unless the context clearly indicates otherwise.

All of the features and/or steps disclosed in the specification can be combined in any combination, except for combinations where at least some of the features and/or steps are mutually exclusive. In particular, preferred features of the invention are applicable to all aspects of the invention and may be used in any combination. The scope of the disclosure is only limited by the appended patent claims.

The invention is further described by the following non-limiting examples.

### Example 1

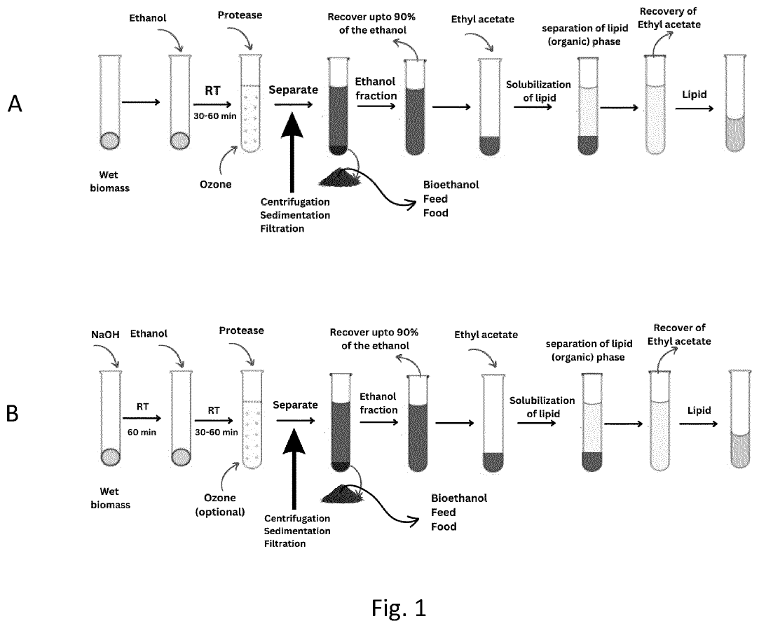

Wet biomass (Chlorella vulgaris, about 75% water) was treated using the process represented schematically in FIG. 1A.

To the wet biomass ethanol (2.5-10 mL ethanol/g biomass) was added and the resulting solution was treated with ozone (1 gm/h, for 10 min). Protease (Bacillus Serine protease, 100 U/mL) was added to the solution after ozone treatment and kept on room temperature shaker (100 rpm) for 60 minutes. The resulting solution was centrifuged to separate precipitated material (protein and carbohydrates) from the soluble fraction.

The soluble ethanol fraction was removed and about 90% of the ethanol removed by distillation. The ethanol thus obtained can be recycled. The remaining concentrated ethanol fraction was extracted with ethyl acetate, and the ethyl acetate subsequently removed by distillation to yield a pure lipid fraction. The removed ethyl acetate can be recycled for further use.

In an alternative process, ethyl acetate was substituted by hexane with overall lipid yield about the same.

Yield: The process resulted in 0.09 g of lipid (dry weight) from 1 g of biomass (dry weight).

### Example 2

The effects of protease treatment in the extraction process described in Example 1 was investigated. In FIG. 2 there is shown the effects of protease on the ethanol treatment step. Thus, treatment with protease (A) results in clear separation of insoluble from soluble material. By comparison, without protease treatment (B) there is inadequate separation of solid material from the liquid ethanol fraction, which would result in lower yield and decreased purity of the final extracted lipid.

### Example 3

As shown by the scheme in FIG. 1b, wet Chlorella vulgaris biomass (1 gm, about 75% water) was resuspended in 5 ml 1 M NaOH solution. After 1 hour, 5 mL ethanol was added and mixed vigorously and left standing for 30 min. The treated biomass was centrifuged and the clear supernatant transferred to new container and ethyl acetate (5 ml) was added, and the mixture was mixed and left to phase separate. The top phase contains the lipid and most of the chlorophyll went to the bottom phase. Over 90% of the chlorophyl is removed from the lipid. The lipid from this process has less than 10% of the chlorophyll present in lipid extracted in FIG. 1a extraction process. A comparison of lipid obtained by this process and lipid obtained without NaOH treatment can be seen if FIG. 3.

### Example 4

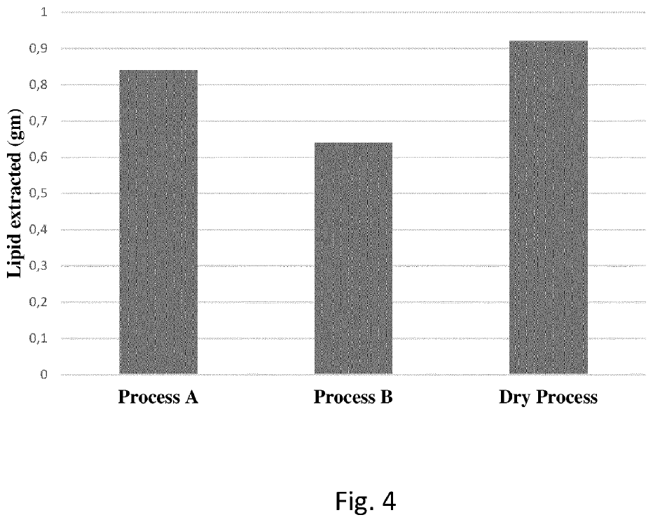

The yields of lipids extracted by the processes shown in FIG. 1 (A) and (B) were compared to the lipid yield by a conventional dry process, where the biomass is dried prior to lipid extraction. As can be seen in the comparison shown in FIG. 4, the yield for process (A) is comparable to that for the conventional dry process, while the yield for process (B) is somewhat lower.

1. A method of lipid extraction for biofuel production, comprising:
a. providing a wet oleogenic microbial biomass;
b. adding ethanol to the wet oleogenic microbial biomass to obtain an ethanol-biomass mixture;
c. treating the ethanol-biomass mixture from step (b) with ozone and/or one or more protease;
d. separating insoluble from soluble material in the ethanol-biomass mixture to obtain an ethanol containing soluble fraction;
e. extracting the ethanol containing fraction with an immiscible organic solvent; and
f. removing the immiscible organic solvent to obtain extracted microalgal lipid.

2. The method of claim 1, wherein step (c) comprises treating the ethanol-biomass mixture with ozone and one or more protease.

3. The method of claim 2, wherein the one or more protease is added to the ethanol-biomass mixture following treatment with ozone.

4. The method of claim 2, wherein the one or more protease is added before, or concomitant with, the treatment with ozone.

5. The method of claim 1, wherein the immiscible organic solvent in step (e) comprises hexane or ethyl acetate or any mixture thereof.

6. The method of claim 1, wherein the ethanol treatment step (b) is preceded by the addition of an alkalic solution containing NaOH or KOH to the oleogenic microbial biomass.

7. The method of claim 1, wherein the oleogenic microbial biomass has a liquid content of 2-10 mL/g biomass.

8. The method of claim 1, wherein the oleogenic microbial biomass is from one or more microalgae.

9. The method of claim 1, wherein the oleogenic microalgal biomass is from one or more psychrophilic microalgae.

10. The method of claim 9, wherein the psychrophilic microalgae are alkaliphilic.

11. The method claim 1, wherein the oleogenic microbial biomass is from one or more genus selected from Chlorella, Nannochloropsis, Chimydomonas, Scenedesmus, Synechocystis, Tetraselmis, Monoraphidium, Ostreococcus, Koliella, Tisochrysis, Pseudopleurochloris sp and Phaeodactylum.

12. The method of claim 1, wherein the oleogenic microbial biomass is from one or more species selected from Chlorella vulgaris, Chimydomonas reinhardtii, Tetraselmis chuii, Tetraselmis suecica, Nannochloropsis gaditana.

13. A process for biofuel production, comprising
a. selecting oleogenic microalgae that have an optimal growth at a temperature of about 20° C. or lower and a pH of 8 or higher;
b. culturing the oleogenic microalgae in an aqueous culture medium in the presence of sunlight, nutrients and CO₂;
c. harvesting oleogenic microalgal cells obtained from the culturing in step (b); and
d. extracting lipids from the harvested oleogenic micoralgal cells obtained in step (c) to produce biofuel.

14. The process of claim 13, wherein the culturing is performed in an open cultivation system such as an open pond, open tank or raceway pond system.

15. The process of claim 13, wherein the extracting in step (d) is performed by a method comprising:
e. providing a wet oleogenic microalgal biomass;
f. adding ethanol to the wet oleogenic biomass to obtain an ethanol-biomass mixture;
g. separating insoluble from soluble material in the ethanol-biomass mixture to obtain an ethanol containing soluble fraction;
h. extracting the ethanol containing fraction with an immiscible organic solvent; and
I. removing the immiscible organic solvent to obtain extracted microalgal lipid.

16. The process of claim 15, wherein following the addition of ethanol in step (f), the ethanol-biomass mixture is treated with ozone.

17. The process of claim 15, wherein the ethanol-biomass mixture obtained in step (f) is further treated with one or more protease.

18. The process of claim 15, wherein the immiscible organic solvent in step (h) is hexane or ethyl acetate.

19. The process of claim 15, wherein the wet micoalgal biomass has a liquid content of 2-10 mL/g biomass.

20. The process of claim 13, wherein the microalgal biomass is from Clamydomonas sp. or Tetraselmis sp.

21-23. (canceled)
